(19) United States
(12) Patent Application Publication
(10) Pub. No.: US 2026/0076532 A1
(43) Pub. Date: Mar. 19, 2026

(54) DEVICES, SYSTEMS, AND METHODS FOR IRRIGATION/ASPIRATION IN A FLUID MANAGEMENT SYSTEM

(71) Applicant: BOSTON SCIENTIFIC SCIMED, INC., Maple Grove, MN (US)

(72) Inventors: Jessica Williams, Marlborough, MA (US); Cassandra Anne Hailson, Marlborough, MA (US); Teagan Jo Walter, Boston, MA (US); Troy Velazquez, Salem, NH (US); Alycia Abbott, Groton, MA (US); Steven T. Carlson, St. Paul, MN (US)

(73) Assignee: BOSTON SCIENTIFIC SCIMED, INC., Maple Grove, MN (US)

(21) Appl. No.: 19/330,238

(22) Filed: Sep. 16, 2025

Related U.S. Application Data
(60) Provisional application No. 63/695,471, filed on Sep. 17, 2024.

(51) Int. Cl. A61B 1/015; A61B 1/00; A61M 3/02

(52) U.S. Cl. CPC A61B 1/015; A61B 1/0004; A61M 3/0201; A61M 3/0202; A61M 3/0208; A61M 3/0216; A61M 3/022; A61M 3/0258; A61M 2205/10; A61M 2205/121; A61M 2205/3334; A61M 2205/3344; A61M 2205/3365; A61M 2205/502

(57) ABSTRACT

A fluid management system including a fluid management console, an outflow pump configured to provide a flow of fluid from a medical device, and a fluid cassette configured to be received within the fluid management console. The console may comprise a housing, a controller housed within the housing, an inflow pump disposed within the housing, and a user input interface. The fluid cassette is configured to be received within a receptacle of the housing of the fluid management console and to provide a flow of fluid to a medical device. The controller of the fluid management console may be configured to adjust a speed of the inflow pump and/or the outflow pump to provide the flow of fluid to the medical device based on one or more control settings including at least a fluid flowrate setting received at the user input interface.

### CROSS-REFERENCE TO RELATED APPLICATIONS

The application claims the benefit of U.S. Provisional Patent Application Ser. No. 63/695,471, filed on Sep. 17, 2024, the disclosure of which is incorporated herein by reference.

### TECHNICAL FIELD

The disclosure is directed to a fluid management system. More particularly, the disclosure is directed to methods and systems for flow control in a fluid management system.

### BACKGROUND

Flexible ureteroscopy (fURS), gynecology, and other endoscopic procedures require the circulation of fluid for several reasons. Surgeons today deliver the fluid in various ways such as, for example, by hanging a fluid bag and using gravity to deliver the fluid, filling a syringe and manually injecting the fluid or using a peristaltic pump to deliver fluid from a reservoir at a fixed pressure or flowrate via a fluid management system. Fluid management systems may adjust the flowrate and/or pressure at which fluid is delivered from the reservoir based on data collected from a procedural device, such as, but not limited to, an endoscope. Of the known medical devices, systems, and methods, each has certain advantages and disadvantages. There is an ongoing need to provide alternative medical devices and fluid delivery systems.

### BRIEF SUMMARY

This disclosure provides design, material, manufacturing method, and use alternatives for components of a fluid management system.

In a first example, a fluid management system may comprise a fluid management console comprising a housing, a controller housed within the housing, an inflow pump disposed within the housing, and a user input interface. The fluid management system may further comprise a fluid cassette configured to be received within a receptacle of the housing of the fluid management console, the fluid cassette configured to provide a flow of fluid to a medical device and an outflow pump configured to provide a flow of fluid from a medical device. The controller of the fluid management console may be configured to adjust a speed of the inflow pump and/or a speed of the outflow pump to provide the flow of fluid to the medical device based on one or more control settings including at least a fluid flowrate setting received at the user input interface.

Alternatively or additionally to any of the examples above, in another example, the one or more control settings may further comprise an intraluminal pressure (ILP).

Alternatively or additionally to any of the examples above, in another example, the one or more control settings may further comprise a flush mode.

Alternatively or additionally to any of the examples above, in another example, the one or more control settings may further comprise a distension mode.

Alternatively or additionally to any of the examples above, in another example, the controller may be configured to increase a speed of the inflow pump while decreasing a speed of the outflow pump to increase an intraluminal pressure.

Alternatively or additionally to any of the examples above, in another example, the controller may be configured to decrease a speed of the inflow pump while increasing a speed of the outflow pump to decrease an intraluminal pressure.

Alternatively or additionally to any of the examples above, in another example, the controller maybe configured to decrease a speed of the inflow pump while decreasing a speed of the outflow pump to decrease a fluid flowrate of the flow of fluid to the medical device.

Alternatively or additionally to any of the examples above, in another example, the controller may be configured to increase a speed of the inflow pump while increasing a speed of the outflow pump to increase a fluid flowrate of the flow of fluid to the medical device.

Alternatively or additionally to any of the examples above, in another example, the controller may be configured to temporarily override the fluid flowrate setting and/or an intraluminal pressure setting to increase a flowrate and/or increase an intraluminal pressure.

In another example, a method for controlling a flow of fluid in a fluid management system may comprise receiving at a user input interface of a fluid management console one or more control settings, activating a fluid management system, and adjusting a speed of a fluid inflow pump configured to provide a flow of fluid to a medical device and/or a speed of an outflow pump configured to provide a flow of fluid from the medical to provide the flow of fluid to the medical device based on one or more control settings including at least a fluid flowrate setting received at the user input interface.

Alternatively or additionally to any of the examples above, in another example, the one or more control settings may further comprise an intraluminal pressure (ILP).

Alternatively or additionally to any of the examples above, in another example, the one or more control settings may further comprise a flush mode.

Alternatively or additionally to any of the examples above, in another example, the one or more control settings may further comprise a distension mode.

Alternatively or additionally to any of the examples above, in another example, the method may further comprise temporarily overriding the fluid flowrate setting and/or an intraluminal pressure setting to increase a fluid flowrate to the medical device and/or increase an intraluminal pressure.

Alternatively or additionally to any of the examples above, in another example, adjusting the speed of the fluid inflow pump may comprise increasing a speed of the fluid inflow pump.

Alternatively or additionally to any of the examples above, in another example, a method for controlling a flow of fluid in a fluid management system may comprise receiving at a user input interface of a fluid management console one or more control settings, activating a fluid flow at a fluid management system, and adjusting a speed of a fluid inflow pump configured to provide a flow of fluid to a medical device and/or a speed of an outflow pump configured to provide a flow of fluid from the medical to provide the flow of fluid to the medical device based on one or more control settings including at least a fluid flowrate setting received at the user input interface. At least one of the one or more control settings may be temporarily overridden in response to activation of a secondary control setting.

Alternatively or additionally to any of the examples above, in another example, the one or more control settings may comprise an intraluminal pressure (ILP).

Alternatively or additionally to any of the examples above, in another example, the secondary control setting may comprise a flush mode.

Alternatively or additionally to any of the examples above, in another example, the secondary control setting may comprise a distension mode.

Alternatively or additionally to any of the examples above, in another example, in response to activation of the flush mode, adjusting the speed of the fluid inflow pump and/or the speed of the outflow pump may comprise increasing a speed of the fluid inflow pump and maintaining or increasing a speed of the outflow pump.

Alternatively or additionally to any of the examples above, in another example, in response to activation of the distension mode adjusting the speed of the fluid inflow pump and/or the speed of the outflow pump may comprise increasing a speed of the fluid inflow pump and maintaining or decreasing a speed of the outflow pump.

Alternatively or additionally to any of the examples above, in another example, adjusting the speed of the fluid inflow pump may comprise increasing a speed of the fluid inflow pump.

The above summary of some embodiments is not intended to describe each disclosed embodiment or every implementation of the present disclosure. The Figures, and Detailed Description, which follow, more particularly exemplify some of these embodiments.

### BRIEF DESCRIPTION OF THE DRAWINGS

The disclosure may be more completely understood in consideration of the following detailed description in connection with the accompanying drawings, in which:

FIG. 1 is a perspective view of an exemplary console of a fluid management system;

FIG. 2 is a perspective view of a fluid management system including the console of FIG. 1 with a disposable fluid tubing set;

FIG. 3 is a schematic view of an illustrative medical device that may be used in conjunction with the fluid management system of FIGS. 1-2;

FIG. 4 is a flow chart of an illustrative method for controlling an irrigation fluid; and

FIG. 5 is an illustrative user interface for use with the fluid management system of FIGS. 1-5.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or”unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar structures in different drawings are numbered the same. The drawings, which are not necessarily to scale, depict illustrative embodiments and are not intended to limit the scope of the disclosure.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device.

Some fluid management systems for use in flexible ureteroscopy (fURS) procedures (e.g., ureteroscopy, percutaneous nephrolithotomy (PCNL), benign prostatic hyperplasia (BPH), transurethral resection of the prostate (TURP), etc.), gynecology, and other endoscopic procedures may regulate body cavity pressure when used in conjunction with an endoscope device such as, but not limited to, a LithoVue^{™}Elite endoscope device using pressure and/or temperature data from the endoscope or other endoscopic device. Direct regulation of the intraluminal pressure during a medical procedure may allow the fluid management system to safely drive pump pressures of up to 600 mmHg to ensure no loss of flow during the procedure when tools are inserted into the working channel of the endoscope device. Irrigation flowrate and intraluminal pressure (ILP) may affect the efficacy and safety of many urological procedures. While the present disclosure is described with respect to urological procedures, the systems and methods described herein may be used in other anatomies, as desired. Irrigation flowrate may be linked to visibility and temperature management while high or low intraluminal pressures may cause adverse events, such as, but not limited to, tissue rupture or working space collapse. In some cases, flowrate and ILP may be controlled indirectly via a set pressure and/or by a hanging saline bag, a pressure cuff, and/or an automated irrigation system. Suction ureteral access sheaths and/or scopes may allow for higher flowrates and lower intraluminal pressures by increasing the pressure differential driving flow out of the kidney. The translation between the pressure on the irrigation system and the pressure on the vacuum pump to a flowrate and ILP may be linked to or correlate to the clinical set-up. Clinical set-up may include the presence or absence of a ureteral access sheath, a size of a ureteral access sheath, a position of a ureteral access sheath, a size of the endoscope (e.g., inner diameter of a working channel, outer diameter of the shaft, or the like), the presence and/or size of a tool in the working channel, and the like. The variability in clinical set-up may result in an inconsistent and/or unpredictable flowrate and/or ILP. The present disclosure is directed towards a fluid management system which allows clinicians to directly set a flowrate and/or ILP for a specific procedure.

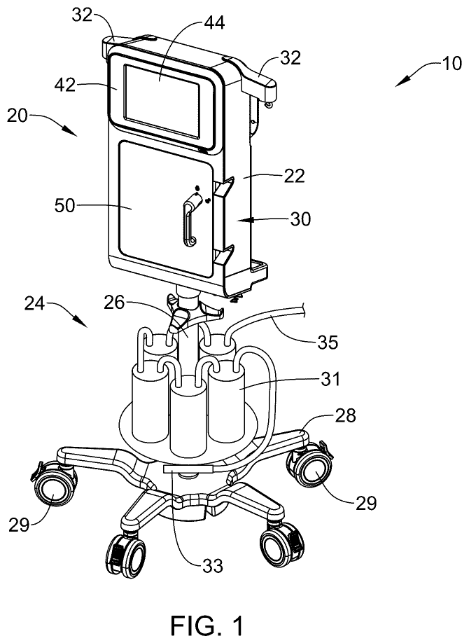

FIG. 1 is a schematic view of a fluid management system 10 that may be used in an endoscopic procedure, such as fURS procedures. The fluid management system 10 may be coupled to a medical device (not shown), such as an endoscope, that allows flow of fluid therethrough. As noted above, in some instances the endoscope may include a pressure sensor, such as the LithoVue™ Elite endoscope, or other endoscope. In some instances, the endoscope may include a temperature sensor to provide intraluminal temperature feedback to the fluid management system 10, a pressure sensor to provide intraluminal pressure feedback to the fluid management system 10, and/or a camera to provide visual feedback to the fluid management system 10.

The fluid management system 10 also includes a fluid management unit or console 20 including a controller 30 housed within a housing 22 of the console 20. In some instances, the console 20 may be portable and/or mobile such that the console 20 may be moved as desired. For instance, the console 20 may be mounted on a wheeled cart 24. For example, the wheeled cart 24 may include a pole 26 extending upward from a base 28 including a plurality of wheels 29 (e.g., caster wheels). In other instances, the console 20 may be provided with another form of cart, configured to be positioned on a flat surface, mounted to a wall, etc.

The fluid management system 10 may also include one or more user input interface components such as a touch screen interface 42. The touch screen interface 42 includes a display screen 44 and may include switches or knobs in addition to touch capabilities. In some embodiments, the controller 30 may include the touch screen interface 42 and/or the display screen 44. The user input interface, e.g., touch screen interface 42, allows the user to input/adjust various functions of the fluid management system 10 such as, for example flowrate, pressure, and/or temperature. The user may also configure parameters and alarms, information to be displayed, and the procedure mode. The user input interface, e.g., touch screen interface 42, allows the user to add, change, and/or discontinue the use of various modular systems within the fluid management system 10. The user input interface, e.g., touch screen interface 42, may also be used to change the fluid management system 10 between automatic and manual modes for various procedures. It is contemplated that other systems configured to receive user input may be used in place of or in addition to the touch screen interface 42 such as, but not limited to, voice commands.

The touch screen interface 42 may be configured to include selectable areas like buttons and/or may provide a functionality similar to physical buttons as would be understood by those skilled in the art. The display screen 44 may be configured to show icons related to modular systems and devices included in the fluid management system 10. The display screen 44 may also include a fluid flowrate and/or fluid pressure display. In some embodiments, operating parameters may be adjusted by touching a corresponding portion of the touch screen interface 42. The touch screen interface 42 may also display visual alerts and/or audio alarms if parameters (e.g., flowrate, temperature, etc.) are above or below predetermined thresholds and/or ranges. In some embodiments, the fluid management system 10 may also include further user interface components such as an optional foot pedal, a fluid warmer user interface, a fluid control interface, or other devices to manually control various modular systems. For example, an optional foot pedal may be used to manually control flowrate. Some illustrative display screens 44 and other user interface components are described in commonly assigned U.S. Patent Application Publication No. 2018/0361055, titled AUTOMATED FLUID MANAGEMENT SYSTEM, the entire disclosure of which is hereby incorporated by reference.

The user input interface, e.g., touch screen interface 42, may be operatively connected to or a part of the controller 30. The controller 30 may be a CPU, including a computer, tablet computer, or other processing device. The controller 30 may be operatively connected to one or more system components such as, for example, an inflow pump, an outflow or vacuum pump, a fluid warming system, and a fluid deficit management system. In some embodiments, these features may be integrated into a single unit. The controller 30 is capable of and configured to perform various functions such as calculation, control, computation, display, etc. The controller 30 is also capable of tracking and storing data pertaining to the operations of the fluid management system 10 and each component thereof. In some embodiments, the controller 30 may include wired and/or wireless network communication capabilities, such as ethernet or Wi-Fi, through which the controller 30 may be connected to, for example, a local area network. The controller 30 may also receive signals from one or more of the sensors of the fluid management system 10. In some embodiments, the controller 30 may communicate with databases for best practice suggestions and the maintenance of patient records which may be displayed to the user on the display screen 44.

The fluid flowrate or the fluid pressure of fluid provided by the fluid management system 10 at any given time may be displayed on the display screen 44 to allow the operating room (OR) visibility for any changes. If the OR personnel notice a change in fluid flowrate or fluid pressure that is either too high or too low, the user may manually adjust the fluid flowrate or the fluid pressure back to a preferred level. The fluid management system 10 may also monitor and automatically adjust the fluid flowrate or the fluid pressure based on previously set parameters.

An illustrative fluid management unit may include one or more fluid container supports, such as fluid supply source hangers 32, each of which may support a fluid supply source (e.g., fluid bag). In some embodiments, placement and/or weight of the fluid supply source(s) hanging from the fluid supply source hanger(s) 32 may be detected using a remote sensor and/or a supply load cell associated with and/or operatively coupled to each fluid supply source hanger 32 and/or fluid container support. The controller 30 may be in electronic communication with the supply load cell. The fluid supply source hanger(s) 32 may be configured to receive a variety of sizes of the first fluid supply source(s) such as, for example, 1 liter (L) to 5 L fluid bags (e.g., saline bags). It will be understood that any number of fluid supply sources may be used. The fluid supply source hanger(s) 32 may extend from the housing 22 of the console 20 and may include one or more hooks from which one or more fluid supply sources may be suspended. In some embodiments, the fluid used in the fluid management unit may be 0.9% saline. However, it will be understood that a variety of other fluids of varying viscosities, concentrations, mixtures, and/or consistencies may be used depending on the procedure.

In some embodiments, the fluid management unit may include one or more collection containers 31, for collecting waste fluid during a medical procedure. The collection containers 31 (e.g., canisters) may be in fluid communication with a vacuum or outflow pump 33 to provide suction for drawing fluid into the collection containers 31. The vacuum pump 33 may be operatively and/or electronically connected to the controller 30. In some embodiments, the vacuum pump 33 may be disposed within the fluid management system 10. Other configurations are also contemplated. In some embodiments, the collection container(s) 31 may be operatively coupled to a collection load cell to detect placement and/or weight of fluid in the collection container(s) to contribute to a fluid deficit calculation. The collection container(s) 31 may be fluidly connected to the medical device via a flexible aspiration tubing 35. The aspiration tubing 35 may be fluidly connected to the medical device in a number of different configurations. For example, the aspiration tubing 35 may be fluidly connected to an aspiration port on the medical device, to an access sheath disposed over the medical device, to a tool configured to be inserted into the working channel of the medical device, etc.

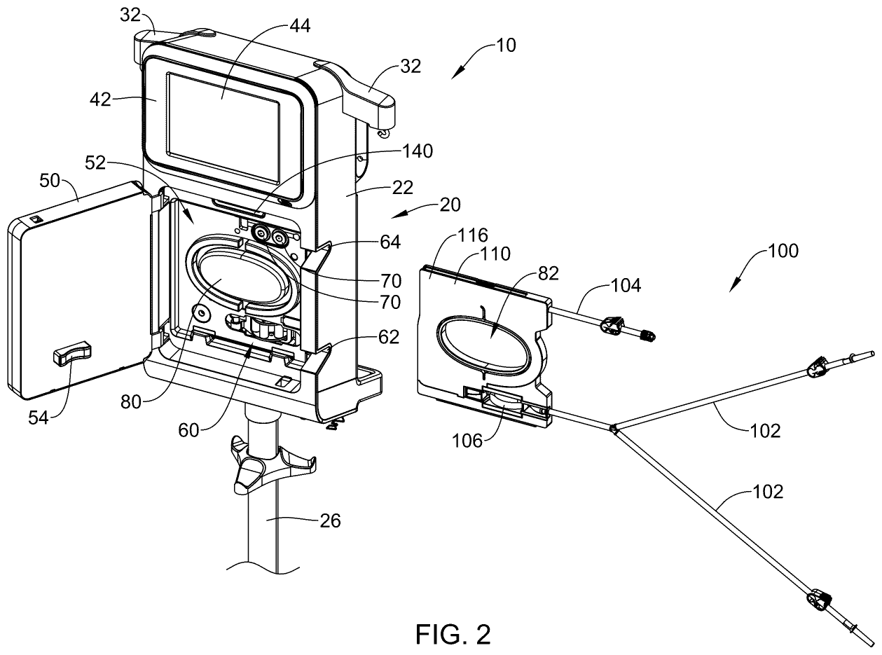

The console 20 may include a door 50 hingedly attached to the housing 22 of the console 20. As shown in FIG. 2, the door 50 may be opened to access a receptacle 52 configured to receive a fluid cassette 110 of a single use fluid tubing set 100 therein. The fluid management system 10 may include an inflow pump 60 configured to operatively engage the fluid tubing set 100 to pump and/or transfer fluid from a fluid supply source (e.g., a fluid bag, etc.) through the fluid tubing set 100 to a treatment site during a medical procedure. For example, the inflow pump 60 may be a roller pump or peristaltic pump positioned in the receptacle 52 configured to engage a length of flexible pump tubing 106 of the fluid cassette 110 when inserted therein. The door 50 may include an occlusion bed 54 mounted on the interior surface of the door 50. The occlusion bed 54 is configured to engage the length of flexible pump tubing 106 of the fluid cassette 110 when the door 50 is closed, to compress the length of flexible pump tubing 106 between the occlusion bed 54 and the inflow pump 60. The occlusion bed 54 may include a concave surface configured to engage the length of flexible pump tubing 106, which extends in an arcuate path around the inflow pump 60.

The inflow pump 60 may be electrically driven and may receive power from a line source such as a wall outlet, an external or internal electrical storage device such as a disposable or rechargeable battery, and/or an internal power supply. The inflow pump 60 may operate at any desired speed sufficient to deliver fluid at a desired pressure such as, for example, 5 mmHg to 50 mmHg, and/or at a target fluid flowrate or a target fluid pressure. The inflow pump 60 may be automatically adjusted based on, for example, pressure and/or temperature readings within the treatment site and/or visual feedback from the medical device attached thereto and inserted into the treatment site. In some embodiments, the controller 30 may be configured to control the inflow pump 60 to maintain a target or predetermined fluid flowrate or target fluid pressure based on a set of system operating parameters. In some embodiments, the controller 30 may be configured to control the inflow pump 60 to maintain a desired fluid pressure at the treatment site or a predetermined flowrate based on a set of system operating parameters.

The inflow pump 60 may also be manually adjusted via, for example, an optional foot pedal, the touch screen interface 42, voice commands, or a separate fluid controller. While not explicitly shown, the fluid controller may be a separate user interface including buttons that allow the user to increase or decrease the inflow pump 60. Alternatively, the fluid controller may be incorporated into the controller 30 and receive input via the touch screen interface 42, voice commands, or other means of input. It will be understood that any number of pumps may be used. In some embodiments, the fluid management system 10 may include multiple pumps having different flow capabilities. In some embodiments, a flow meter may be located before and/or after the inflow pump 60.

The fluid management system 10 may be user selectable between different modes based on the procedure, patient characteristics, etc. For example, different modes may include, but are not limited to, fURS Mode, BPH Mode, Hysteroscopy Mode, Cystoscopy Mode, etc. Once a mode has been selected by the user, mode parameters such as fluid flowrate, fluid pressure, fluid deficit, and temperature may be provided to the user via the display screen. The exemplary parameters of the specific modes may be previously determined and loaded onto the controller 30 using, for example, software. Thus, when a user selects a procedure from an initial display on the touch screen interface display screen 44, these known parameters may be loaded from the controller 30 to the various components of the fluid management system 10. The fluid management system 10 may also be user selectable between automatic and manual mode. For example, for certain procedures, the user may wish to manually adjust a fluid flowrate, fluid pressure, and/or other parameters. Once the user has selected the manual mode on, for example, the touch screen interface 42, the user may then adjust fluid flowrate or fluid pressure via other manual interfaces such as an optional foot pedal, voice commands, or the fluid control interface. If the user selects an automatic mode, the user may be prompted to select or input via the touch screen interface 42 which medical device (e.g., endoscope) is being used so that the controller 30 may determine if data obtained from the medical device can be used to facilitate control of the fluid management system 10. In some embodiments, the fluid management system 10 may be configured to verify the medical device (e.g., endoscope) selected is actually being used prior to using the collected data.

The single use tubing set 100 may include inflow tubing 102 providing a fluid inflow from the fluid supply source into the interior of the fluid cassette 110. In some instances, the inflow tubing 102 may include a bifurcated tubing with a first tubing section fluidly connected to a first fluid supply source and a second tubing section fluidly connected to a second fluid supply source. The first and second tubing sections may converge (such as at a Y-fitting) to a common tubing section extending to the fluid cassette 110. The end of the first tubing section and/or the second tubing section may include a bag spike, or other connector, for connecting to the fluid supply source(s). The single use tubing set 100 may also include outflow tubing 104 providing a fluid outflow from the interior of the cassette 110 to a medical device connected thereto. The single use tubing set 100, including the fluid cassette 110, the inflow tubing 102, and the outflow tubing 104, may be disposable and provided sterile and ready to use.

When the fluid cassette 110 is installed in the receptacle 52 and the door 50 is closed, the inflow tubing 102 may pass through a channel 62 extending through a wall of the housing 22 of the console 20 to an exterior of the console 20. Likewise, when the fluid cassette 110 is installed in the receptacle 52 and the door 50 is closed, the outflow tubing 104 may pass through a channel 64 extending through a wall of the housing 22 of the console to an exterior of the console 20. The channel 62 and the channel 64 may both extend from the exterior of the console 20 to the receptacle 52. In some instances, both the channel 62 and the channel 64 may be located on the same sidewall of the console 20 such that both the inflow tubing 102 and the outflow tubing 104 extend from the console 20 on the same side of the console 20.

In some embodiments, the fluid management system 10 may include a fluid warming system 80, as shown in more detail in FIG. 2, for heating fluid to be delivered to the patient. The fluid warming system 80 may be an inductive heating system in some instances. In other instances, the fluid warming system 80 may be an infrared fluid warming system. Other fluid warming system configurations and methods may also be used, as desired. For example, the fluid warming system 80 may include one or more heat sources such as, for example a platen system or an inline coil in the fluid supply line to heat the fluid using electrical energy. Fluid warming may be specifically designed and tailored to the flowrates required in the specific application of the fluid management system 10. Some illustrative fluid warming systems are described in commonly assigned U.S. Patent Application Publication No. 2018/0361055, titled AUTOMATED FLUID MANAGEMENT SYSTEM, the entire disclosure of which is hereby incorporated by reference.

The fluid warming system 80 may include a heater configured to interact with the fluid cassette 110 to heat fluid passing therethrough. When the fluid cassette 110 is coupled with the heater, a susceptor positioned in the fluid path of the cassette 110 may be positioned within an induction coil of the fluid warming system 80 and be configured to heat the fluid flowing through or past the susceptor as the fluid passes through the fluid flow path of the cassette 110.

While not explicitly shown, the fluid warming system 80 may include a heater user interface included with or separate from the touch screen interface 42. In one example, the heater user interface may simply be a display screen providing a digital display of the temperature of the fluid entering and/or exiting the susceptor in the fluid flow path of the cassette 110. In another embodiment, the user interface may also include temperature adjustment buttons to increase or decrease the temperature of the fluid exiting the cassette 110. In this embodiment, the heater user interface and/or the display screen may indicate the current temperature of the fluid exiting the cassette 110 as well as the target temperature to be reached. It is noted that all information output from the fluid warming system 80 may be transmitted directly to the display screen 44 such that no heater user interface is necessary.

The fluid warming system 80 may include one or more sensors configured to monitor the fluid flowing therethrough. For example, temperature sensors may be mounted in the fluid warming system 80 such that they detect the temperature of the fluid flowing through the fluid cassette 110. In some embodiments, a first temperature sensor may be located at or near the fluid inlet to the susceptor and/or the fluid outlet from the susceptor so that they detect the temperature of fluid flowing through the fluid cassette 110 prior to the fluid entering the susceptor and after fluid exits the susceptor. In some embodiments, additional sensors may be located at a medial portion of the susceptor so that they detect a progression of temperature increase of the fluid in the fluid cassette 110.

The console 20 may further include one or more additional sensors, such as a pressure sensor and/or a bubble sensor. For instance, the console 20 may include a pressure sensor 70, illustrated as a pair of pressure sensors, configured to monitor a system pressure (i.e., pump pressure) of fluid exiting the cassette 110 and flowing through the outflow tubing 104 to a surgical site. The fluid cassette 110 may include a corresponding pressure sensor interface (not explicitly shown), such as a flexible membrane, that allow the pressure sensor 70 to monitor the pressure of fluid flowing through the fluid cassette 110 when the fluid cassette 110 is installed in the receptacle 52 of the console 20. The pressure sensor 70 may send information to the controller 30 and/or display screen 44. Additional features of the cassette 110 of the fluid tubing set 100 are described in commonly assigned U.S. Patent Application No. 63/640,089, titled DEVICES, SYSTEMS, AND METHODS FOR FLOW COMPENSATION IN A FLUID MANAGEMENT SYSTEM, the entire disclosure of which is hereby incorporated by reference.

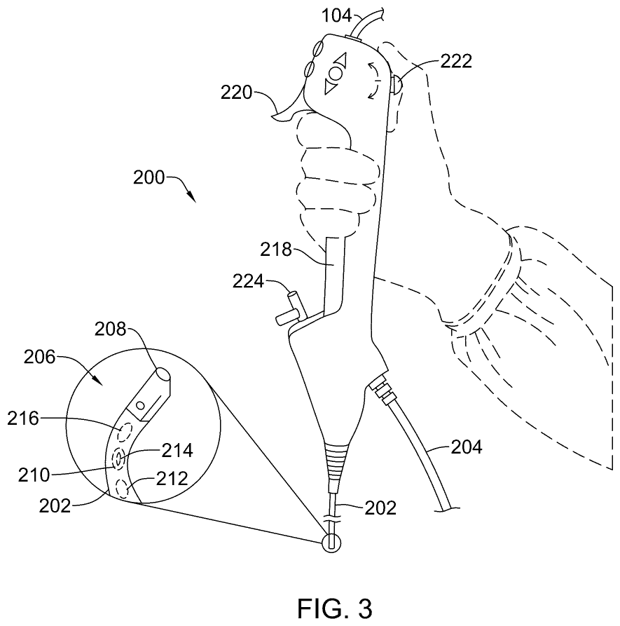

FIG. 3 illustrates aspects of a medical device 200 that may be used in conjunction with the fluid management system 10. In the illustrated embodiments, the medical device 200 may be a ureteroscope such as a LithoVue™ Elite endoscope, another intraluminal pressure sensing endoscope, or other endoscope. However, other medical devices, such as another endoscope, may be used in addition to or in place of a ureteroscope. The medical device 200 may be configured to deliver fluid from the fluid management system 10 to the treatment site via an elongate shaft 202 configured to access the treatment site within the patient. In some embodiments, the inflow pump 60 may be in fluid communication with the elongate shaft 202. The elongate shaft 202 may include one or more working lumens for receiving a flow of fluid or other medical devices therethrough. The medical device 200 is connected to the fluid management system 10 via one or more supply line(s) 104 (e.g., a tube), as shown in FIG. 2 for example.

In some embodiments, the medical device 200 may be in electronic communication with a workstation (not explicitly shown) via a wired connection 204. The workstation may be in wired or wireless communication with the controller 30 of the fluid management system 10. In some embodiments, the workstation may be a multi-use component (e.g., used for more than one procedure) while the medical device 200 may be a single use device, although this is not required. In some embodiments, the workstation may be omitted and the medical device 200 may be electronically coupled directly to the controller 30 of the fluid management system 10.

As shown in FIG. 3, the medical device 200 may include one or more sensors proximate a distal end 206 of the elongate shaft 202. For example, the medical device 200 may include a pressure sensor 208 at a distal tip of the elongate shaft 202 to measure intraluminal pressure within the treatment site. The medical device 200 may also include other sensors such as, for example, a temperature sensor 210, a Fiber Bragg grating optical fiber 212 to detect stresses, and/or an antenna or electromagnetic sensor 214 (e.g., a position sensor). In an illustrative embodiment, the distal end 206 of the medical device 200 may also include at least one camera 216 to provide a visual feed to the user on the display screen of the workstation. In another embodiment, the medical device 200 may include two cameras 216 having different communications requirements or protocols so that different information may be relayed to the user by each camera 216. When so provided, the user may switch back and forth between cameras 216 at will through the touch screen interface 42 and/or the workstation. While not explicitly shown, the elongate shaft 202 may include one or more working lumens for receiving the fluid and/or other medical devices.

The medical device 200 includes a handle 218 coupled to a proximal end of the elongate shaft 202. The handle 218 may have a fluid flow on/off switch 220, which allows the user to control when fluid is flowing through the medical device 200 and into the treatment site. The handle 218 may further include other buttons 222 that perform other various functions. For example, in some embodiments, the handle 218 may include buttons to control the temperature of the fluid. It will be understood that while the exemplary embodiment describes a ureteroscope, the features detailed above may also be directly integrated into a cystoscope, an endoscope, a hysteroscope, or virtually any device with an image capability. In some embodiments, the medical device 200 may also include a drainage port 224 which may be connected to a drainage system such as the vacuum pump 33 and the collection containers 31. Some illustrative drainage systems are described in commonly assigned U.S. Patent Application Publication No. 2018/0361055, titled AUTOMATED FLUID MANAGEMENT SYSTEM, the disclosure of which is hereby incorporated by reference.

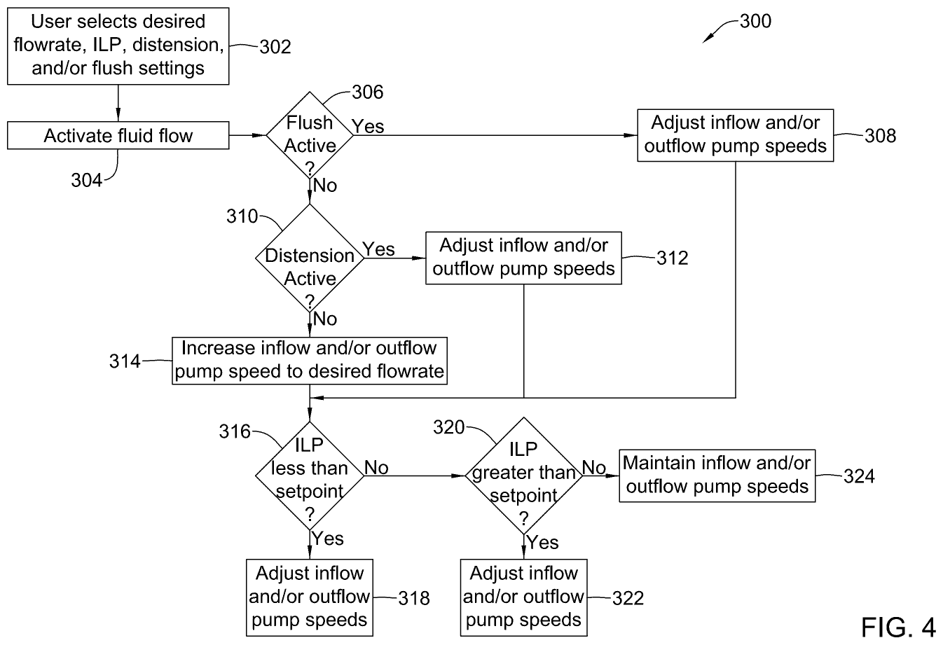

The inflow pump 60 and the outflow pump 33 may be controlled to allow the clinician to directly set a flowrate and/or an intraluminal pressure (ILP) for a particular procedure. The fluid management system 10 may then be controlled to maintain the desired flowrate and/or ILP. For example, the controller 30 may be configured to control a pump speed of both the inflow pump 60 and the outflow pump 33 to achieve a desired flowrate and/or ILP. FIG. 4 is an illustrative flow chart of a method 300 for controlling the inflow pump 60 and the outflow pump 33 to achieve a desired setting. Generally, the speeds of the inflow pump 60 and/or the outflow pump 33 may be determined based on a user-selected flowrate, ILP, and/or flush/distension settings. The order of the steps shown in FIG. 4 is not limiting. For example, the steps may be rearranged or performed in an alternative order, as desired. In some cases, one or more of the steps may be performed substantially simultaneously with one or more other steps. To begin, the clinician may enter or select desired settings for the fluid management system 10, as shown at block 302. The settings may be entered at the user interface 42. However, the clinician may select or enter the control parameters remotely using a remote device or at a different controller. Some illustrative control settings may include, but are not limited to, flowrate, ILP, flush, and/or distension.

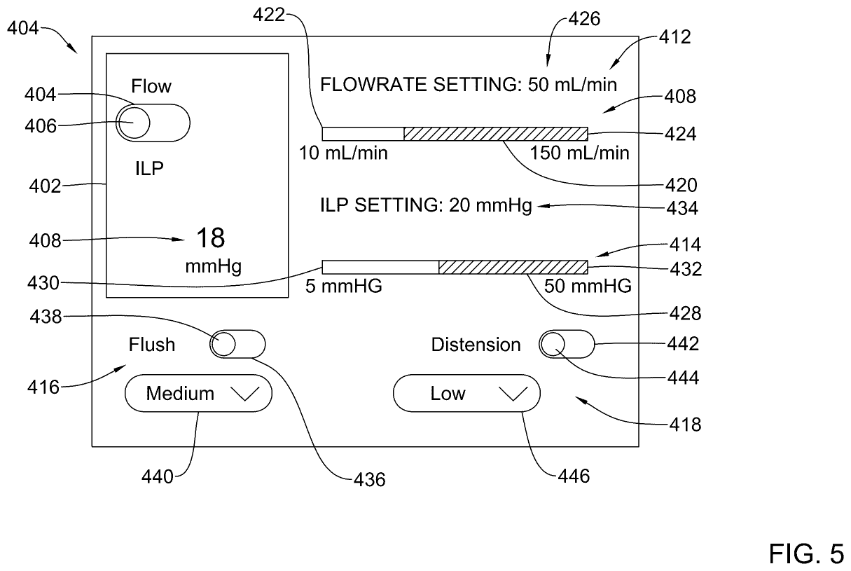

Referring briefly to FIG. 5, which depicts an illustrative guided user interface (GUI) 400 for entering control parameters, the adjustable parameters may be presented to the user. In some cases, the GUI 400 may include a region 402 for displaying the current operating conditions. For example, the region 402 may include a toggle display 404 which may both indicate whether or not flow is active and allow the user to turn flow on/off. The toggle display 404 may be configured to display a first color when flow is active and a second color when flow is inactive. Further, the toggle display 404 may include a button icon 406 which moves between the left side and the right side of the toggle display 404 in a similar manner to a switch when the flow is turned on and/or off. It is contemplated that the GUI 400 may be configured to display the flow status with words (e.g., on/off) or colors (e.g., red/green), display an actual flowrate, or the like. One of skill in the art will recognize there are many different ways to visually display the status of the fluid flow. The current operating conditions region 402 may be further configured to display the ILP 408. In some embodiments, the ILP 408 may be measured at a pressure sensor (e.g., pressure sensor 208 in FIG. 3). The ILP 408 may be displayed as a numerical value. However, other representative display methods may be used as desired, such as, but not limited to color gradients, slide bars, graphs, or the like.

The GUI 400 may further include a parameter adjustment region 410. The parameter adjustment region 410 may include a flowrate setting region 412, an ILP setting region 414, a flush setting region 416, and a distension setting region 418. In some cases, the GUI 400 may allow the user to select or enter desired setpoints for additional parameters, if so desired. Further, one or more of the parameter setting regions 412, 414, 416, 418 may be omitted. The flowrate setting region 412 may allow the user to enter or select a desired flowrate. In the illustrated example, the flowrate setting region 412 may include a slide bar 420 showing a minimum flowrate 422 and a maximum flowrate 424. In some cases, the minimum flowrate 422 and/or the maximum flowrate 424 may be displayed alphanumerically. In one illustrative example, the minimum flowrate 422 may be 10 milliliters per minute (mL/min) and the maximum flowrate 424 may be 150 mL/min. This is just an example. The minimum flowrate 422 may be less than 10 mL/min and the maximum flowrate 424 may be greater than 150 mL/min. The user may move the slide between the minimum flowrate 422 and a maximum flowrate 424 to the desired flowrate. In some cases, the user may be prompted to enter a numerical value for the desired flowrate. In another example, the flowrate may be selectable from a dropdown menu. In yet another example, the flowrate may be selectable through actuation of icons or buttons configured to increase or decrease the flowrate. These are just some examples. The flowrate setting region 412 may be further configured to display the current flowrate setting 426. The current flowrate setting 426 may be displayed alphanumerically to allow the user to easily see the current setpoint.

The ILP setting region 414 may allow the user to enter or select a desired ILP. In the illustrated example, the ILP setting region 414 may include a slide bar 428 showing a minimum ILP 430 and a maximum ILP 432. In some cases, the minimum ILP 430 and/or the maximum ILP 432 may be displayed alphanumerically. The user may move the slide between the minimum ILP 430 and a maximum ILP 432 to the desired ILP. In one illustrative example, the minimum ILP 430 may be 5 millimeters of mercury (mmHg) and the maximum ILP 432 may be 50 mmHg. This is just an example. The minimum ILP 430 may be less than 5 mmHg and the maximum ILP 432 may be greater than 50 mmHg. In some cases, the user may be prompted to enter a numerical value for the desired ILP. In another example, the ILP may be selectable from a dropdown menu. In yet another example, the ILP may be selectable through actuation of icons or buttons configured to increase or decrease the flowrate. These are just some examples. The ILP setting region 414 may be further configured to display the current ILP setting 434. The current ILP setting 434 may be displayed alphanumerically to allow the user to easily see the current setpoint.

The flush setting region 416 may allow the user to temporarily increase the fluid flowrate or “flush” the treatment region. For example, the flush may be turned on and off at a toggle display 436 which may both indicate whether or not flow is active and allow the user to turn the flush on/off. The toggle display 436 may be configured to display a first color when flush is active and a second color when flush is inactive. Further, the toggle display 436 may include a button icon 438 which moves between the left side and the right side of the toggle display 436 in a similar manner to a switch when the flush is turned on and/or off. It is contemplated that the GUI 400 may be configured to display the flush status with words (e.g., on/off) or colors (e.g., red/green), or the like. One of skill in the art will recognize there are many different ways to visually display the status of the flush. In some embodiments, the GUI may include a dropdown menu 440 configured to allow the user to select a relative intensity of the flush. For example, the dropdown menu 440 may allow the user to select between “low”, “medium”, and “high” flush settings, where “high” increases the flowrate to the greatest extent. Other flush settings, such as, but not limited to, numerical settings and/or a desired flush duration, may be used as desired. In some cases, when flush is off, the dropdown menu may be deactivated or shaded.

The distension setting region 418 may allow the user to temporarily increase the ILP or “distend” the treatment region. For example, the distension setting may be turned on and off at a toggle display 442 which may both indicate whether or not distension is active and allow the user to turn the distension on/off. The toggle display 442 may be configured to display a first color when distension is active and a second color when distension is inactive. Further, the toggle display 442 may include a button icon 444 which moves between the left side and the right side of the toggle display 442 in a similar manner to a switch when distension is turned on and/or off. It is contemplated that the GUI 400 may be configured to display the distension status with words (e.g., on/off) or colors (e.g., red/green), or the like. One of skill in the art will recognize there are many different ways to visually display the status of the distension. In some embodiments, the GUI may include a dropdown menu 446 configured to allow the user to select a relative intensity of the distension. For example, the dropdown menu 446 may allow the user to select between “low”, “medium”, and “high” distension settings, where “high” increases the ILP to the greatest extent. Other distension settings, such as, but not limited to, numerical settings and/or a desired distension duration, may be used as desired. In some cases, when distension is off, the dropdown menu may be deactivated or shaded.

Returning to FIG. 4, once the user inputs or selects the desired control settings, the fluid flow may be turned on or activated, as shown at block 304. In one example, the fluid flow may be activated through actuation of the toggle button 406. The controller 30 may be configured to continually update the operation of the inflow pump 60 and the outflow pump 33 as the control settings are changed. For example, the controller 30 may be configured to determine if the flush setting is active, as shown at block 306. The flush setting may be a secondary control setting. If the user has turned on flush (for example, through actuation of the toggle button 438), the controller 30 may be configured to adjust the speeds of the inflow pump 60 and/or outflow pump 33, as shown at block 308. When flush is active, more fluid is needed within the patient, however it may be desirable for the ILP to remain constant. Thus, when flush is active, the pump speed of the inflow pump 60 and the speed of the outflow pump 33 may be increased proportionally to one another to increase a volume of fluid entering the patient without increasing the ILP. When flush is turned off, or deactivated, the controller 30 may be configured to verify the inflow pump 60 and outflow pump 33 are operating to maintain the flowrate and/or the ILP at the setpoint values.

If flush is not active, the controller 30 may be configured to determine if the distension setting is active, as shown at block 310. The distension setting may be a secondary control setting. If the user has turned on distension (for example, through actuation of the toggle button 444), the controller 30 may be configured to adjust the speeds of the inflow pump 60 and/or outflow pump 33, as shown at block 312. When distension is active, the ILP may be increased while maintaining a desired flowrate. The pump speed of the inflow pump 60 and the speed of the outflow pump 33 may be increased disproportionately to increase a pressure within the patient's body. For example, the speed of the inflow pump may be increased more than the speed of the outflow pump 33 such that more fluid is entering the body than leaving the body. When distension is turned off, or deactivated, the controller 30 may be configured to verify the inflow pump 60 and outflow pump 33 are operating to maintain the flowrate and/or the ILP at the setpoint values.

It is contemplated that when flush or distension are active, the controller 30 may be configured to override the setpoint flowrate to achieve flush or distension. If distension or flush is not active, the controller 30 may be configured to increase or decrease the speed of the inflow pump 60 and/or the outflow pump 33 to achieve the desired flowrate, as shown at block 314. The speeds of the inflow pump 60 and/or outflow pump 33 may be set to achieve fluid balance in the system such that a similar volume of fluid is entering and leaving the patient. It is contemplated that the controller 30 may be configured to determine a flowrate of the fluid entering the patient based on the speed of the inflow pump 60. Alternatively, or additionally, the controller 30 may be configured to determine a flowrate of the fluid entering the patient based on the pressure of fluid flowing through the fluid cassette 110, as measured at pressure sensor 70.

While the fluid flow is activated, the controller 30 may also be configured to adjust the speed of the inflow pump 60 and/or outflow pump 33 to achieve the setpoint ILP. The controller 30 may be configured to compare a current or measured ILP to the setpoint ILP to determine if the measured ILP is less than the setpoint ILP, as shown at block 316. The measured ILP may be received at the controller 30 from the pressure sensor 208 located adjacent the distal end of the endoscope 200. If the measured ILP is less than the setpoint ILP, the speeds of the inflow pump 60 and/or the outflow pump 33 may be adjusted to increase the ILP, as shown at block 318. To increase the ILP, the volume of fluid entering the patient may be greater than the volume of fluid exiting the patient. To increase the ILP, the speed of the inflow pump 60 may be increased to be greater than the speed of the outflow pump 33. Alternatively, or additionally, the speed of the outflow pump 33 may be decreased. In some cases, the speed of the inflow pump 60 may increase while the speed of the outflow pump 33 is simultaneously decreased. If the measured ILP is not less than the setpoint ILP, the controller 30 may be configured to compare a current or measured ILP to the setpoint ILP to determine if the measured ILP is greater than the setpoint ILP, as shown at block 320. If the measured ILP is greater than the setpoint ILP, the speeds of the inflow pump 60 and/or the outflow pump 33 may be adjusted to decrease the ILP, as shown at block 322. To decrease the ILP, the volume of fluid entering the patient may be less than the volume of fluid exiting the patient. To decrease the ILP, the speed of the inflow pump 60 may be decreased to be less than the speed of the outflow pump 33. Alternatively, or additionally, the speed of the outflow pump 33 may be increased. In some cases, the speed of the inflow pump 60 may be decreased while the speed of the outflow pump 33 is simultaneously increased. If the measured ILP is neither greater than or less than the setpoint ILP (e.g., if the measured ILP is equal to the setpoint ILP), the controller 30 may be configured to control the speeds of the inflow pump 60 and the outflow pump 33 to maintain the ILP at the setpoint ILP. To maintain a constant ILP, the inflow pump 60 and the outflow pump 33 may be operated at a speed such that a volume of fluid entering the patient is approximately the same as the volume of fluid exiting the patient. It is contemplated that the speeds of the inflow pump 60 and/or outflow pump 33 needed to maintain steady state may depend on a size of the respective pump and/or a type of pump. For example, the inflow pump 60 and the outflow pump 33 may be operated at similar speeds or different speeds from one another to achieve fluid balance or steady state.

It is contemplated that the present system may allow fluid to be introduced into the body and removed from the body in a manner which eliminates or minimizes air leaks. This may allow the system 10 to respond to control setting changes quicker than if air leaks are present.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A fluid management system, comprising:
a fluid management console, comprising:
a housing;
a controller housed within the housing;
an inflow pump disposed within the housing; and
a user input interface;
a fluid cassette configured to be received within a receptacle of the housing of the fluid management console, the fluid cassette configured to provide a flow of fluid to a medical device; and
an outflow pump configured to provide a flow of fluid from a medical device;
wherein the controller of the fluid management console is configured to adjust a speed of the inflow pump and/or a speed of the outflow pump to provide the flow of fluid to the medical device based on one or more control settings including at least a fluid flowrate setting received at the user input interface.

2. The fluid management system of claim 1, wherein the one or more control settings further comprises an intraluminal pressure (ILP).

3. The fluid management system of claim 1, wherein the one or more control settings further comprises a flush mode.

4. The fluid management system of claim 1, wherein the one or more control settings further comprises a distension mode.

5. The fluid management system of claim 1, wherein the controller is configured to increase a speed of the inflow pump while decreasing a speed of the outflow pump to increase an intraluminal pressure.

6. The fluid management system of claim 1, wherein the controller is configured to decrease a speed of the inflow pump while increasing a speed of the outflow pump to decrease an intraluminal pressure.

7. The fluid management system of claim 1, wherein the controller is configured to decrease a speed of the inflow pump while decreasing a speed of the outflow pump to decrease a fluid flowrate of the flow of fluid to the medical device.

8. The fluid management system of claim 1, wherein the controller is configured to increase a speed of the inflow pump while increasing a speed of the outflow pump to increase a fluid flowrate of the flow of fluid to the medical device.

9. The fluid management system of claim 1, wherein the controller is configured to temporarily override the fluid flowrate setting and/or an intraluminal pressure setting to increase a flowrate and/or increase an intraluminal pressure.

10. A method for controlling a flow of fluid in a fluid management system, the method comprising:
receiving at a user input interface of a fluid management console one or more control settings;
activating a fluid management system; and
adjusting a speed of a fluid inflow pump configured to provide a flow of fluid to a medical device and/or a speed of an outflow pump configured to provide a flow of fluid from the medical to provide the flow of fluid to the medical device based on one or more control settings including at least a fluid flowrate setting received at the user input interface.

11. The method of claim 10, wherein the one or more control settings further comprises an intraluminal pressure (ILP).

12. The method of claim 10, wherein the one or more control settings further comprises a flush mode.

13. The method of claim 10, wherein the one or more control settings further comprises a distension mode.

14. A method for controlling a flow of fluid in a fluid management system, the method comprising:
receiving at a user input interface of a fluid management console one or more control settings;
activating a fluid flow at a fluid management system; and
adjusting a speed of a fluid inflow pump configured to provide a flow of fluid to a medical device and/or a speed of an outflow pump configured to provide a flow of fluid from the medical to provide the flow of fluid to the medical device based on one or more control settings including at least a fluid flowrate setting received at the user input interface;
wherein at least one of the one or more control settings is temporarily overridden in response to activation of a secondary control setting.

15. The method of claim 14, wherein the one or more control settings comprises an intraluminal pressure (ILP).

16. The method of claim 14, wherein the secondary control setting comprises a flush mode.

17. The method of claim 14, wherein the secondary control setting comprises a distension mode.

18. The method of claim 16, wherein in response to activation of the flush mode adjusting the speed of the fluid inflow pump and/or the speed of the outflow pump comprises increasing a speed of the fluid inflow pump and maintaining or increasing a speed of the outflow pump.

19. The method of claim 17, wherein in response to activation of the distension mode adjusting the speed of the fluid inflow pump and/or the speed of the outflow pump comprises increasing a speed of the fluid inflow pump and maintaining or decreasing a speed of the outflow pump.

20. The method of claim 14, wherein adjusting the speed of the fluid inflow pump comprises increasing a speed of the fluid inflow pump.
